(19) United States
(12) Patent Application Publication
(10) Pub. No.: US 2011/0281295 A1
(43) Pub. Date: Nov. 17, 2011

(54) METHOD AND DEVICE FOR CULTURING ALGAE

(75) Inventor: Julien Sylvestre, Paris (FR)

(73) Assignee: PHOTOFUEL SAS, Paris (FR)

(21) Appl. No.: 13/146,025

(22) PCT Filed: Jan. 26, 2010
(86) PCT No.: PCT/EP10/50878

§ 371 Date: Jul. 25, 2011

(30) Foreign Application Priority Data
Jan. 27, 2009 (FR) 09-00352

(51) Int. Cl. C12Q 1/02; C12N 1/12; A01G 1/00; C12M 1/00

(52) U.S. Cl. 435/29; 435/292.1; 435/257.1; 47/1.4

(57) ABSTRACT

According to one aspect, the invention relates to a device for culturing algae with natural light, including an enclosure with a culturing medium and the algae to be cultured, and a substrate arranged to receive solar radiation in order to perform photoconversion of said solar radiation, the substrate including at least one luminescent compound making it possible to reemit radiation having a spectrum adapted to the optimisation of a biological parameter of interest resulting from the photosynthesis of said algae.

### FIELD OF INVENTION

The invention relates to a method and device for the cultivation of algae.

### STATE OF THE ART

In this document, “algae” refers, by convenience, to any kind of microscopic aquatic photosynthetic organism such as microalgae, cyanobacteriae, microscopic angiosperms (“micro-crops” such as duckweed).

These algae can be obtained from the hundreds of thousands of species naturally present on the earth surface, or have been genetically modified using techniques known to those skilled in the art.

Algae can be grown as pure cultures (a single species) or as mixed cultures containing several different algae species, identified or not.

Algae can be grown in fresh water, sea water or brackish water, clean or used.

Algae can be cultivated per se or in order to fabricate a diversity of chemical compounds (cellulose, sugars, alcohols, lipids, proteins) by recycling carbon dioxide as organic water via the reaction of photosynthesis. This chemical compounds can be produced inside the algae cells or secreted.

When the chemical compounds of interest are not secreted, the cultivated algae are separated from the water that contains them, continuously or using batch processes, by various methods known to those skilled in the art.

In a direct fashion, or after conversion, some of the produced or secreted chemical compounds of interest can be integrated into various products or supplements for the chemical industries (e.g. ethanol), food and feed (e.g. omega 3), cosmetics or pharmacy. Some of those chemical compounds can be used to manufacture biofuels such as bioethanol, biodiesel and a variety of “designer fuels” that can be directly substituted, partially or totally, to gasoline, diesel or jet fuel used in road, rail, air and sea transportation.

Algae can also, by various methods known to those skilled in the art, be used to produce biohydrogen or bioelectricity.

Biorefineries built around algae cultivation therefore provide several important advantages in the domains of chemistry, energy, environment, alimentation and health compared to existing processes that rely on fossil-based substrates. There are three ways to grow algae: without light, with artificial light or with solar light.

### Heterotrophic Algae Cultivation

Algae cultivation in the absence of light is similar to fermentation and hence uses apparatuses and technologies that are adapted from well-established fermentation industries. This approach, however, has two major drawbacks.

First, qualitatively, it is estimated that a small minority of about 1 to 10% of algae in nature can be adapted to this type of heterotrophy.

Second, from a quantitative point of view, the substrate typically used for fermentation, is sugar. But the annual world sugar production for all uses, including alimentation and ethanol production, is 170 million tons per year, representing, at 17 kJ per g, an amount of energy of about 2.9 10̂18 Joules. World energy consumption being 500 Exajoules (5 10̂20 Joules), and without even considering non-unity conversion yields, one sees that this heterotrophic approach is unable to substitute, at large scale, for fossil fuels used to produce the majority of the energy currently used for human activities and productions. Worldwide energy consumption can also be accounted for in metric tons of oil equivalent, at 12.2 10̂9 tons (at 45 kJ per g), which again is incomparable with the total sugar produced in the world now, at 0.17 10̂9 tons (with an energy density of only 17 kJ per g). Finally, algae cultivation in heterotrophy requires a prior step of photosynthesis allowing the fixation of organic matter (sugars and possibly other substrates) by terrestrial plants. This step requires large amount of fertilizers, water, energy, human labor and soils and its environmental balance is very imperfect.

### Algae Cultivation Under Artificial Light

Algae are cultivated, in the laboratory, in the presence of artificial light. One can imagine cultivating microalgae at large scale using a variety of devices that incorporate artificial light sources. When the object of algae cultivation is biofuels, it is easy to see, however, that the cost of this approach is prohibitive. We confine ourselves here to the marginal cost of electricity to produce artificial light, without taking into account the capital and maintenance cost of the algae cultivation system or the cost of installing and replacing light sources. We take a low cost of electricity, at 5 cts per kWh, a yield of 20% for the artificial light, a global yield of 25% for this source of light (eight photons are needed to create a “CH₂O” molecule), an oil content of the algae equal to 50% (in mass). We consider that the oil extracted from the algae has an energy density close to that of diesel, at 12 kWh per kg. Producing 1 kg of oil implies, under these very optimistic assumptions, a marginal cost of electricity of

12*0.05/(0.20*0.25*0.5)=24 euros per kg

or more than 20 times the price ever reach for petroleum, and without even taking into accounts all the other fixed and variable cost factors, that are naturally very significant. We therefore observe that algae cultivation in the presence of artificial light is reserved to the production of high-value compounds, not basic alimentary, chemical or energetic compounds.

### Algae Cultivation in the Presence of Sunlight

The annual average power of sunlight radiation received on the earth surface is around 90,000 TW. Human energy consumption is equivalent to an average power in the order of 18 TW, i.e., 5,000 times less. It is therefore obvious that sunlight constitutes a renewable primary energy source that is more than enough abundant to meet all human energy needs. Photosynthesis allows, with instantaneous yields measured between 0.02 and 10% and more generally average yields observed between 0.1 and 2%, to directly convert sunlight energy into biofuels and bioproducts, and hence into a source of energy or chemical compounds of good value because storable and modifiable, which solar thermal or photovoltaic approaches do not allow, as they simply produce electricity and heat, which are difficult to store and therefore need to be utilized as soon as they are produce, which does not match with the peak consumption periods.

Regarding vegetable oil production by photosynthesis, it is known that algae offer much higher surfacic yields than traditional land-based crops such as colza or even palm oil (between 5 and 100 times). In addition to that, algae do not require agricultural land, which eliminates the problems set forward for current biofuels, of competition with food and negative impact of land use change and agricultural practices such as deforestation. Algae can finally allow direct recycling of concentrated carbon dioxide and wastewater.

Thus, various authors calculate that a non-agricultural area (land or sea) with a size of a small European country could be enough to ensure the production of a large fraction of the energy, chemicals and food used by man. In a recent article (K. M. Weyer et al. <<Theoretical maximum of algae oil cultivation>>, Bioenerg. Res. (2009) DOI 10.1007/s12155-009-9046-x), the authors have calculated, for the first time in detail, the maximum surfacic annual yield obtainable when growing algae under sunlight. They obtain a result between c. 50,000 L. ha⁻¹. year⁻¹ (<<practical maximum>>) and c. 350,000. L. ha⁻¹. year⁻ (“theoretical maximum”, for which all yields are set to 100%). In the discussion of the various factors contributing to the yield calculation, the authors isolate three (terms 4, 5 and 8)—photon transmission efficiency, photon utilization efficiency and biomass accumulation efficiency—which, according to them, have a potential to be improved. The proposed improvements, joining those of other authors in the field, concern the geometry of the cultivation system, to favour light distribution, as well as the choice of algae, in particular in terms of their tolerance to high light intensities.

These authors take for granted the second term of the calculation (immediately after the first term which is received solar energy), the PAR (photosynthetically active radiation), which is the fraction of sunlight usable for photosynthesis, conventionally taken as wavelengths between 400 and 700 nm i.e., 45.8% of solar energy received on the earth surface.

It appears that algae cultivation in the presence of sunlight is the only technique offering a potential for large-scale development under economically and environmentally acceptable conditions. However, this technique has a limitation related to its yield.

The applicant has shown that prior art devices work with a suboptimal use of solar energy and that it is possible to significantly increase the yield. Thus, the invention concerns a method and device that improve the yield of algae cultivation. This yield improvement positively impact economic viability and environmental balance (life-cycle analysis), in particular for biorefineries.

### SUMMARY OF THE INVENTION

In a first aspect, the invention relates to a device for algae cultivation under natural light comprising an enclosure with a cultivation medium and algae to cultivate, wherein said device comprises additionally a substrate to receive solar radiation in order to photo convert said solar radiation, said substrate comprising at least one luminescent compound enabling the reemission of a radiation whose spectrum is adapted to the optimization of a biological parameter of interest resulting from the said algae photosynthesis.

According to a first variant, the substrate is interposed between incident solar radiation and the enclosure.

According to a second variant, the enclosure is formed of a cultivation pool, covered at least partially by the substrate.

According to a third variant, the substrate constitutes a wall of the enclosure.

Advantageously, the enclosure is formed by a circuit of tubes in which circulates the cultivation medium containing the algae in suspension.

In a particular embodiment, the enclosure is made of a flexible bag constituting the substrate, made of a significantly transparent material doped with at least one luminescent compound.

According to another embodiment, the substrate includes particles suspended in the cultivation medium, one or several luminescent compounds being incorporated within the particles.

Preferentially, the substrate comprises at least two luminescent compounds.

In a preferred embodiment, the absorption spectrum of at least one of said luminescent compounds at least partially overlaps the emission spectrum of at least one of said luminescent compounds.

Advantageously, at least one of said luminescent compounds has an absorption spectrum covering the 300-360 nm wavelength band and an emission spectrum covering the 340-400 nm band.

In an embodiment, at least one of said luminescent compounds emits according to an anti-Stokes mechanism.

In an embodiment, the device integrates a CO2 source.

In an embodiment, the device comprises, in addition, a concentrator of solar energy.

In a particular embodiment, the said luminescent compounds have absorption or emission spectra that promote algae photosynthesis.

In a second aspect, the invention relates to a fabrication process for an algae cultivation device according to the first aspect comprising:
The prior exposure of said algae to be cultivated to a variety of wavelengths;
Measurement, for each said wavelengths of a biological parameter of interest and assessment, for said parameter of one or several adapted wavelengths;
Selection of one or several luminescent compounds enabling the photoconversion of sunlight at said adapted wavelengths;
Production of a substrate containing said one or several selected luminescent compounds.

In a variant, said biological parameter of interest is the growth speed of algae.

In another variant, said biological parameter of interest is oil production by algae.

In another variant, said biological parameter of interest is the production of a given pigment by algae.

In a third aspect, the invention concerns a method for the cultivation of algae under natural light comprising the setting of a culture of algae in an enclosure with a cultivation medium, wherein said process comprises the photoconversion of sunlight by a substrate containing at least one luminescent compound that emits radiation whose spectrum is adapted to the photosynthesis of said algae.

Compared to the calculations by K. M. Weyer et al. mentioned above, the methods of the invention lead to a 20% to 100% or more increase of both theoretical and practical maxima, at a limited capital and operational cost increase, therefore bringing a major economic benefit. This technique is compatible with numerous cultivation systems and with other more classical improvements proposed by a diversity of authors, in particular the use of algae strains that have been selected or genetically modified.

### BRIEF DESCRIPTION OF THE DRAWINGS

Other characteristics and advantages of the invention will appear when reading the following description, illustrated by figures representing:

FIG. 1, a luminescent substrate integrated in the cover of a cultivation pond,

FIG. 2, a luminescent substrate integrated in the walls of a photobioreactors,

FIG. 3, a luminescent substrate dispersed in a cultivation medium.

### DETAILED DESCRIPTION OF THE INVENTION

The invention concerns methods and devices to modify sunlight enabling to overcome the limit of the PAR (photosynthetically active radiation) as presented above.

The applicant has shown that it is precisely by raising the PAR value above the 45.8% value conventionally accepted by all authors, which is not suggested in the literature, that the method of the invention allows with a very high generality and adaptability and for a limited cost, to improve the yield of algae cultures and to exceed the maximum currently accepted by specialists. The invention is applicable to a large number of algae cultivation systems including photobioreactors and simpler pond-type systems.

Indeed, the concept of PAR is a simplification as some wavelengths in the PAR range—such as green (interval around 550 nm) for green algae—do not lead to an efficient photosynthesis. PAR is therefore an overestimate of the fraction of solar radiation that is effectively converted. The method of the invention enables to modify sunlight in order to reduce those poorly efficient wavelengths of PAR in favour of more efficient wavelengths.

The applicant has shown that by a fine spectral modulation of incident sunlight to match the photosynthetic requirement of the alga or algae cultivated, the method of the invention provides an original and quantitatively important improvement that is applicable to almost all sunlight-dependent algae cultivation processes that are described or envisioned in the literature.

In its most general sense, the invention concerns a device for the cultivation of algae under natural light, comprising an enclosure with a cultivation medium and algae to be cultivated, wherein the device additionally comprises a substrate set forward to receive solar radiation in order to enable the photoconversion of said solar radiation, the substrate comprising at least one luminescent compound allowing to reemit a radiation whose spectrum is adapted to the optimization of a biological parameter of interest resulting from the photosynthesis of said algae.

The luminescent compounds used are preferentially fluorescent organic dyes and preferentially laser dyes. One chooses molecules with a high quantum yield (higher than 0.6, preferentially higher than 0.9), a low cost and a good stability after exposure to UV-visible light (over several months, preferentially several years).

The luminescent compounds used can include rare earth compounds such as terbium or europium salts.

The luminescent compounds used can include inorganic compounds such as quantum dots.

The luminescent compounds used can be chosen from the following group of compounds:

Group A—emission at 340 nm
PPO diphenyloxazole (Lambda Physic Lambdachrome LC3700)
POPOP (Lambdachrome LC 4230)
DPS (Lambdachrome LC4090)
QUI P-quinqaphenyl (Lambdachrome LC3690)
   Group B—emission around 400-460 nm
8S OB thiophenediylbis benzoxazole
Coumarin (several Lambdachrome references)
   Group C—emission around 560 nm
Hostasole 3G naphtalimide (Clariant)
Lumogen 083 perylene (BASF)
Rhodamine 110 (Lambdachrome)
   Group D—emission around 580-640 nm
Hostazole GG thioxanthene benzanthione (Clariant)
Lumogen 305 peylene (BASF)
Rhodamine (Lambdachrome)
6G ethylaminoxanthene
   Group E—emission around 640-680 nm
Cretsyl violet diaminobenzole
Sulforhodamine B (Lambdachrome LC6200)

### Group F—700-1000 nm

Rhodamine 800 (Sigma)
Pyridine 2 (Lambdachrome LC7600)
DOTC-HITC (Lambdachrome LC7880)
Styril 9 (Lambdachrome LC8400)

In addition to the cited molecules, numerous molecules of “laser dyes” type known to those skilled in the art and in particular several molecules named Coumarin or Rhodamin (various suppliers), as well as molecules from the Lumogen Dyes series (BASF/ColorFlex) are usable.

Preferentially, a combination of several luminescent compounds comprising a compound from group A such as PPO or two compounds from group A such as PPO and OB, as well as a compound from each of the 1 to 3 following groups (B, C, D, E or F) is used.

Concentrations used vary preferentially between 0.1 and 1,000 ppm, preferentially between 1 and 100 ppm.

Preferentially, a system of absorption-reemission in series is used wherein the wavelength of maximum emission from one compound corresponds to the wavelength of maximum absorption of the next compound in the series. This facilitates FRET (fluorescence resonance energy transfer) phenomena that are favorable from an energy point of view. For example, a group A compound, a group B compound and a group C compound are used.

Depending on the concentration used and the physic-chemical properties of the matrix in which the luminescent compounds are integrated, one can observe the apparition of excimer (“excited dimers”) type phenomenon which modified, in a favorable fashion, the optical properties of the compounds taken at a monomer state. The concentrations that favor the apparition of excimers in a given matrix are sought and can be determined empirically.

Preferentially, when several luminescent compounds are used, the concentrations used decrease when the excitation wavelength increases. This non-trivial concentration rule is inspired by what is observed within phycobilisomes and enable to limit auto-absorption phenomena.

The substrate within which the luminescent compounds are integrated is a plastic, for example acrylic a plastic such as polymethylmethacrylate (PMMA) or ethylene vinyl acetate (EVA), Apoliah (Arkema), polyvinylidene fluoride (PVDF), polyethylene (PE) or polycarbonate (PC).

According to a variant, the luminescent compounds are integrated within a resin or coating that is spread on plates or glass tubes.

In a variant, TiO₂ nanoparticles and/or aluminum oxide at 0.1-0.5% by weight are integrated within the substrate and play the role of a light diffuser and UV reflector.

In a particular embodiment, the luminescent compounds are incorporated within millimeter-sized polystyrene beads, made of PMMA or of another polymer, and are suspended within the cultivation medium. Advantageously, those beads also allow cleaning the walls of the enclosing the culture and avoiding their dirtying. The beads constitute an illumination source internal to the cultivation medium and allow lifting a limitation of prior art devices regarding the distribution of light within the cultivation volume.

In a variant, said millimeter-sized beads are rendered phosphorescent by employing compounds luminescing over a long period of time (>10³ s). The fluorescent beads, which circulate within the cultivation medium, have been previously illuminated either by sunlight, or by an UV flash or another source of high energy monochromatic artificial light. Such beads can be used at night or days. They can contain ZnS crystals with 10-1000 ppm copper or silver dopants.

In a variant, one of the luminescent compounds used is chosen by a person skilled in the art in order to convert, by an anti-Stokes mechanism, a fraction of infrared radiation (700-2 000 nm) into visible radiation, preferentially red radiation (600-700 nm).

Complementary benefits brought by the method according to the invention are described in a non-limitative fashion below:

### Advantage Linked to the Creation of a Diffuse Light

The use of luminescent compounds according to the invention leads to a reemission of incident sunlight in all directions of the space. In practice, a given luminescent compound reemits incident light in an anisotropic fashion (with a “doughnut”-shaped distribution) but the orientation of said compound within the material that transforms the light, which is itself random, leads to a statistically isotropic reemission at 4 pi steradians. This allows to transform incident sunlight into a diffuse light. The diffuse light so obtained is favorable to the growth of algae, as it limits photoinhibition phenomena.

Also, the method of the invention promotes algae growth when the algae are illuminated by direct sunlight or when the light that illuminates the algae is itself diffuse, which is the case when weather conditions include clouds and/or water vapor.

### Advantage Linked to the Limitation of Ultraviolet Radiation

The method of the invention absorbs a fraction of sunlight UV (260-400 nm) and reemits it into visible wavelength (400 nm and more). This allows to limit the exposure of algae to UVs, whom people known in the art know they can limit the growth of said algae and even, in some cases, lead to mutations that can render genetically inhomogeneous and finally destabilize the cultivated species.

### Thermal Advantages

The modification of sunlight operated by the method of the invention leads to an advantageous modification of the temperature profile to which the cultivated algae are exposed. The effect depends on the chosen cultivation device (photobioreactor, greenhouse, bag or open pond) but it combines, with more or less intensity, on the one hand a decrease in the average and maximum values of daily temperatures and an increase in the average and minimum values of night temperatures. These two thermal effects increase the average productivity of the algae cultures, Moreover, they reduce the occurrence of extreme temperature conditions which are not favorable and can lead to the extinction of algae cultures having been exposed to abnormally hot or cold temperatures.

### Application to the Case of Green, Red, and Brown Algae and to Cyanobacteriae.

The method of the invention allow to modify sunlight to adapt it to the needs of a diversity of algae species.

Green algae harbor a photosynthetic apparatus that makes photosynthesis particularly efficient in the presence of blue (440 nm) and red (680 nm) lighting. The method of the invention allows, by using a combination of appropriate luminescent compounds, to modify natural sunlight whose spectrum displays a single maximum around 550 nm in order to obtain a light whose spectrum displays two maxima, one around 440 nm and a second one around 680 nm. For example, a group A compound, a group B compound and a group D compound are used.

Contrary to green algae, red algae poorly use blue light and red light but reproduce with a maximum efficiency when illuminated by green light, around 560 nm. The method of the invention allows, by using a combination of appropriate luminescent compounds, to modify sunlight spectrum in order to increase the intensity of its maximum around 550 nm and decrease the intensity of one or several other regions of the spectrum. For example, a compound from group A, a compound from group B and a compound from group C are used.

Contrary to algae, cyanobacteriae do not have chloroplasts. Those prokaryotic cells have a specific photosynthetic apparatus and a specific pigment content, which leads them to grow in an optimal fashion when they are illuminated by a type of light that is enriched in wavelength comprised between 580 and 650 nm. The method of the invention allows to enrich sunlight between 580 and 650 nm by converting wavelength smaller than 580 nm (and/or wavelength over 650 nm). For instance, a group A compound, a group B compound, a group E compound and a group F compound are used.

It is however important to note that the relation between the photosynthetic pigments or accessory pigment content, the absorption spectrum of the culture and the photosynthetic action spectrum is not always simple or well understood (in some instances, some of the wavelengths ranges in which the isolated pigments or the algae culture absorb efficiently do not correspond to a high photosynthetic yield). An empirical measurement of the action spectrum is therefore generally preferable and presented below.

A privileged embodiment is described below, that allows to increase the productivity of a given algae species.

Beyond the general specifications indicated above, it can be advantageous in practice to measure the action spectrum of a particular species.

Step 0—A given algae species that one wished to cultivate is chosen, isolated from its natural habitat or genetically modified.
Step 1—A broadband white light source and a monochromator or a filtered light with a diversity of interferential filters is chosen and adjusted in power so that each color transmit by the filter has the same intensity, or, preferentially, a colored light source is used, for example blue, green, red etc. LEDs of identical intensities. The algae culture is this way exposed to various wavelength ranges.
Step 2—One measures, for each illumination condition, the photosynthetic activity of the algae (for example by measuring the oxygen produced and/or the carbon dioxide consumed) and the average productivity spectrum (g/L/day) is deduced as a function of incident light.
Step 3—A combination of luminescent compounds is selected to modify sunlight, whose spectrum is easy to obtain separately, in order to concentrate it in wavelengths that have been empirically observed to lead to a maximum productivity of algae culture.
Step 4—A combination of luminescent compound whose composition has just been determined at step 3 is incorporated into a masterbatch.
Step 5—Said masterbatch, a monomer and potentially other additives known to people skilled in the art are mixed to create a plastic material.
Step 6—Said doped plastic material is extruded and thus plates, tubes or films are created from which a photobioreactor or a covering element are built that accelerate the growth of algae chosen at step 0.

### Application to Increasing the Production of a Given Compound by an Algae Species

Algae can allow the production of a diversity of compounds of interest such as pigments. Empirically, by realizing several algae cultures with a diversity of wavelength intervals (for instance with twelve LEDs of the same power illuminating in ten different wavelengths intervals comprised between 350 and 950 nm: 350-400 nm, 400-450 nm, 450-500 nm etc.), identify an optimal wavelength interval that leads to a larger quantity of the compound of interest. The compositions of the luminescent compounds used by the method of the invention are subsequently adapted in order to modify sunlight to concentrate it in the optimal interval.

### Application to the Production of Oil

Certain algae species can contain an important quantity of oil, up to 50% or more than their dry mass. However, those skilled in the art have observed that in general, the conditions allowing algae to grow at maximum speed are different from the conditions that allow each cell to accumulate a large quantity of lipids. Often therefore, at an appropriate moment of the production, the cultivation conditions of an algae culture that grows quickly are modified in order to promote, in a second stage, lipid accumulation; Several types of stresses are possible, notably a stress by nitrogen deprivation. The method of the invention offers the possibility to use a modification of light to generate a stress that promotes liquid production. The nature of the spectrum Soil adapted to the generation of said stress can be determined in the laboratory by analyzing the response (lipid content per gram of dry matter) of an algae culture of interest exposed to different wavelengths of artificial light. One can subsequently expose, at an appropriate moment during production, a large-scale culture of the algae of interest, to a form of sunlight modified by the method of the invention in order to obtain a spectrum close to the ideal spectrum Soil previously determined. This allows said culture to accumulate large quantities of lipids.

### Hybrid Cultivation Systems.

The method of the invention is applicable to hybrid cultivation systems that combine the advantages of photobioreactors (controlled environment, high productivity) and those of pools (reduced cost). Thus one can first start an algae pre-culture within photobioreactors that make use, or do not, of AlgoSun technology then transfer said preculture to a pool that uses AlgoSun technology. Different segments of the reactor can contain materials that are doped in different ways.

Examples below illustrate in a non-limitative fashion embodiments of the device according to the invention.

### Example 1

### Tubular Photobioreactors

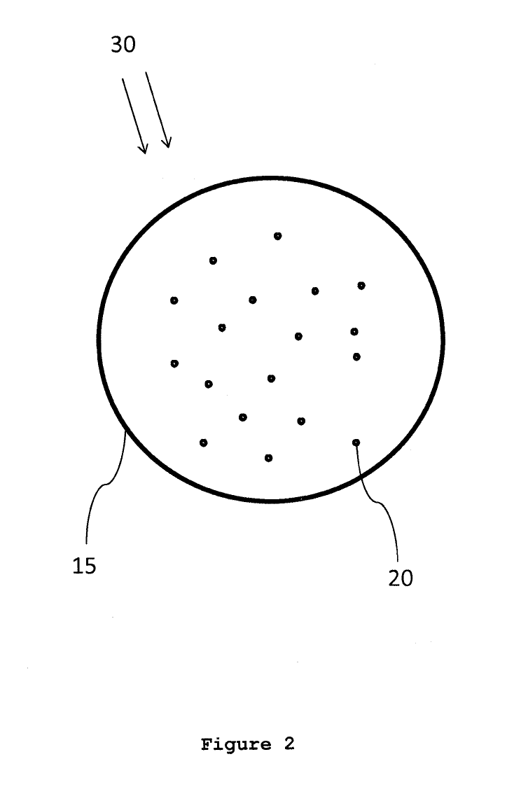

Algae are cultivated in a device containing a network of plastic tubes whose diameter is comprised between 5 and 20 cm and whose total length can reach several km. A side view of part of the device is shown schematically on FIG. 2.? Algae 20 are set to culture within a tubular photobioreactor illuminated by natural light 30. The wall 15 if the photobioreactor receives natural light and is composed of a material that contains at least one luminescent compound allowing the reemission of radiation whose spectrum is adapted to algae. The device integrates pumps and a system to inject concentrated carbon dioxide. The tube plastics is doped, before extrusion, by a combination of luminescent compounds chosen in order to modify sunlight depending on the physiological needs of the algae species considered, as previously determined experimentally. Polymethylmethacrylate (PMMA) is an example of acrylic plastics that offers excellent optical properties and allows a good integration of luminescent compounds and can be utilized for the manufacture of tubes. A PMMA thickness comprised between 1 and 5 mm is used. For green algae, the following formula can be used for 3 mm PMMA plates, for 1 kg of MMA:
0.44 g PPO (Lambdachrome),
0.22 g OB,
0.06 g Rhodamin 800 (Sigma),
0.12 g Cretsyl violet (Clariant).

### Example 2

### Bags

A cheaper solution to cultivate algae consists in using bags. Said bags can be set in open air, in a closed area, or let floating on the sea (preferentially, semi-permeable bags that let water go out and exchange nutrients with sea water are used). The plastic bags can be made of a polymer such as polyethylene-ethylene vinyl acetate (PE-EVA), Apoliah (Arkema) or PMMA. The thickness of the bags is comprised between 100 μm and 500 μm. The plastic is doped, before extrusion, with a combination of luminescent compounds chosen in order to modify sunlight based on the physiological needs of the algae species considered, as previously determined experimentally.

### Example 3

### Shelters

The whole algae cultivation device, which can integrate tubes, parallelepiped volumes or panels, is integrated within a “shelter”-type structure, which can be closed or semi-closed and plays a positive role in terms of thermal regulation, light regulation, protection from parasites, predators or adverse weather. The greenhouse walls are composed of glass whose internal face has been coated with a resin doped by a combination of luminescent compounds chosen in order to modify sunlight according to the physiological needs of the algae species considered, as previously determined experimentally. Alternatively, coated glass can be replaced by PMMA plates.

### Example 4

### Film

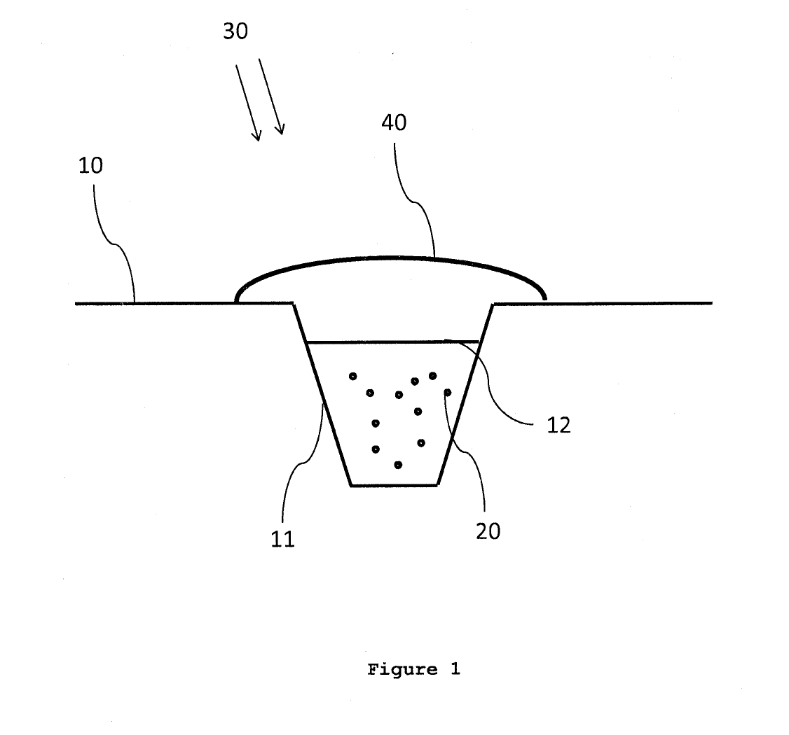

Algae are cultivated in an open air raceway-type or trough-type system. A side view of part of such a cultivation system is displayed at FIG. 1. In the earth 10, a trough 11 acts as an enclosure illuminated by natural light 30 within which algae 20 are suspended in an aqueous medium of larger area 12. Sunlight is received and modified by at least one luminescent compound allowing the reemission of radiation whose spectrum is adapted to algae.

Elements from the pond r troughs are covered by the flexible or slightly rigid film, which can for example be made of a plastic material doped with a combination of luminescent compounds chosen in order to modify sunlight according to the physiological needs of the algae species considered, as previously determined experimentally. The film can be made of PMMA, PE-EVA or PVDF?.

### Example 5

### Millimeter Beads

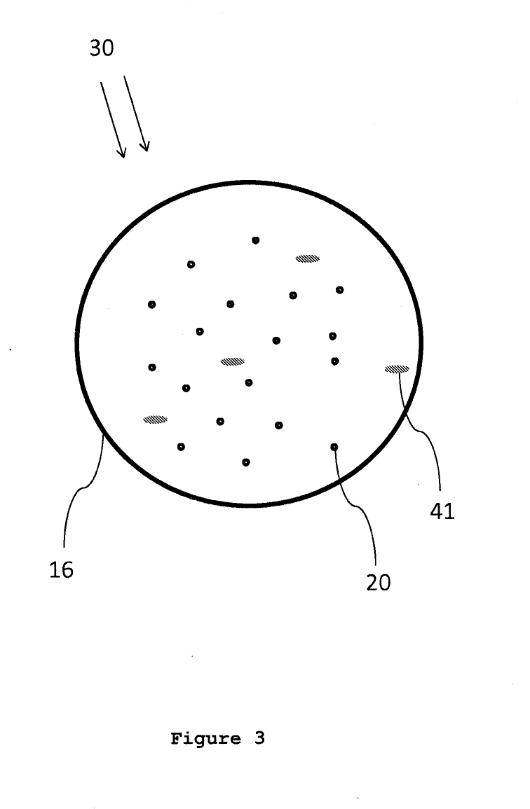

FIG. 3 shows a side view of part of a device according to a variant. A closed tubular photobioreactor system 16 is illuminated by natural light 30 to allow the cultivation of algae 20. Particles 45 containing at least one luminescent compound that emits radiation whose spectrum is adapted to algae are put into the cultivation medium. Particles can for example be made of beads whose diameter is comprised between 1 mm and 5 mm. Said beads can be beads that are classically used to clean the walls of the tubes and avoid the formation of an adhesive layer of algae on the surface of the tubes, which would end up impeding light penetration. The method of the invention gives a new role to said beads, which are doped by short-lived luminescence (fluorescence) or long-lived luminescence (phosphorescence) luminescent compounds, this last instance also allowing to work in full darkness. Thus, the beads, which are constantly agitated by the movement imposed to the photobioreactor fluid, realize, within the same cultivation medium, a spectral adaptation and constitute an internal diffuse light source that promote algae growth within the whole aqueous volume. The beads can be made from polymethylmethacrylate (PMMA), polypropylene, nylon, PVC or polyvinyl acetate. Polystyrene beads or polymethylmethacrylate beads (PMMA) can be used. One can use a phosphor agent to decrease the density of the beads while creating trapping vacuoles or concentrating photons.

Although described through a certain number of examples and detailed embodiments, the device and method of the invention encompasses different variants, modifications and improvements that will appear obvious to a person skilled in the art, and it is assumed that said different variants, modifications and improvements are part of the invention, as defined by the following claims.

1. A device for the cultivation of algae under natural light, comprising:
an enclosure with a cultivation medium and algae to be grown; and
a substrate set forward to receive solar radiation in order to photo convert said solar radiation, said substrate comprising at least one luminescent compound enabling to reemit radiation whose spectrum is adapted to the optimization of a the production of a chemical compound of interest resulting from the photosynthesis of said algae.

2. The device according to claim 1, wherein said substrate is set between incident solar radiation and the enclosure.

3. The device according to claim 2, wherein said enclosure is composed of a cultivation pond at least partially covered by said substrate.

4. The device according to claim 1, wherein said substrate forms a wall of said enclosure.

5. The device according to claim 4, wherein said enclosure is composed of a circuit of tubes within which the cultivation medium and the algae in suspension flow.

6. The device according to claim 4, wherein said enclosure is composed of a flexible bag forming the substrate made of a sensibly transparent material doped with said luminescent compounds.

7. The device according to claim 1, wherein said substrate comprises particles set in suspension in the cultivation medium, the luminescent compounds being incorporated into said particles.

8. The device according to claim 1, wherein the substrate comprises at least two luminescent compounds.

9. The device according to claim 8, wherein the absorption spectrum of at least one of the luminescent compounds at least partially overlaps with the emission spectrum of at least one other luminescent compound.

10. The device according to claim 1, wherein at least one of said luminescent compounds has an absorption spectrum covering the 300-360 nm band and an emission spectrum covering the 340-400 nm band.

11. The device according to claim 1, wherein at least one of said luminescent compounds has an emission that follows an anti-Stokes mechanism.

12. The device according to claim 1, wherein said device further comprises a source of carbon dioxide.

13. The device according to claim 1, wherein said device further comprises a concentrator of incident solar energy.

14. (canceled)

15. A method for optimization of production of a chemical compound of interest using photosynthetic algae, comprising:
prior exposure of algae to be grown to different wavelength intervals;
measurement, for each of said different wavelength intervals, of the production of the chemical compound of interest and the determination of one or several wavelength intervals adapted to said production;
selection of one or several luminescent compounds enabling the photoconversion of sunlight to said adapted wavelength intervals;
manufacture of a substrate comprising said selected luminescent compounds; and
harvest of said algae in a device comprising an enclosure with a cultivation medium and algae to be grown, wherein said substrate is set forward to receive solar radiation in order to photo convert said solar radiation.

16. (canceled)

17. A method according to claim 15, wherein said chemical compound of interest is one selected from a group consisting of an oil, a sugar, a protein, a pigment, and a biogas.

18.-19. (canceled)

20. The device according to claim 1, wherein at least one of said luminescent compounds has an absorption spectrum covering a 260-400 nm band and an emission spectrum in the visible range, above 400 nm.

21. The method according to claim 15, wherein said chemical compound of interest being excreted by said algae, the method further comprises extraction of the chemical compound of interest from the cultivation medium.

22. The method according to claim 15, wherein said chemical compound of interest is produced in said algae, and wherein the method further comprises extraction of the chemical compound of interest from the algae.

23. A method for recycling carbon dioxide using photosynthetic algae comprising:
prior exposure of algae to be grown to different wavelength intervals;
measurement, for each of said different wavelength intervals, of the degradation of said carbon dioxide and the determination of one or several wavelength intervals adapted to said degradation;
selection of one or several luminescent compounds enabling the photoconversion of sunlight to said adapted wavelength intervals,
manufacture of a substrate comprising said selected luminescent compounds,
harvest of said algae in a device comprising an enclosure with a cultivation medium and algae to be grown, wherein said substrate set forward to receive solar radiation in order to photo convert said solar radiation.

24. A method for recycling waste water using photosynthetic algae comprising:
prior exposure of algae to be grown to different wavelength intervals in a cultivation medium comprising waste water,
measurement, for each of said different wavelength intervals, of the degradation of a compound of the waste water and the determination of one or several wavelength intervals adapted to said degradation,
selection of one or several luminescent compounds enabling the photoconversion of sunlight to said adapted wavelength intervals,
manufacture of a substrate comprising said selected luminescent compounds,
harvest of said algae in a device comprising an enclosure with a cultivation medium and algae to be grown, and said substrate set forward to receive solar radiation in order to photo convert said solar radiation.
